(19) United States
(12) Patent Application Publication
(10) Pub. No.: US 2008/0021263 A1
(43) Pub. Date: Jan. 24, 2008

(54) Implantable mechanical device that have the ability to apply traction to organs or anatomical tissues for its reposition during and after a surgical intervention

(76) Inventors: Antoni Castello Escude, Terrassa (Barcelona) (ES); Francisco Farrer Velazquez, Calatayud (Zaragoza) (ES)

Correspondence Address: Ralph A. Dowell of DOWELL & DOWELL P.C., 2111 Eisenhower Ave, Suite 406, Alexandria, VA, 22314, US

(21) Appl. No.: 11/489,460

(22) Filed: Jul. 20, 2006

(51) Int. Cl. A61F 2/00

(52) U.S. Cl. 600/29

(57) ABSTRACT

Implantable mechanical device to apply traction to organs or anatomical tissues for its repositioning during and after a surgical intervention that comprises an implantable box in whose inside housing a drum with a toothed wheel that gears in a worm are housed to wind in a traction threads and a fixation screw for the traction threads fixation once passed through the implantable box; a base support placed under the box to spread the stress per square inch and stabilization of the said box against the supportive tissue; different systems to subject or support organs or anatomical tissues, such as a sling provided with a spread bars to maintain the sling fully open even under traction; and inserting elements to insert the traction system in the patients body.

This invention consist of an “Implantable mechanical device to apply traction to organs or anatomical tissues for its repositioning during and after a surgical intervention”.

It pertains generally to the technical sector of solving urinary incontinence in male and female, supporting or tractioning the rectus to solve fecal incontinence, supporting or tractioning the pelvic floor in patients with prolapsus, supporting or reducing the diameter of the vessels in the vascular system, aneurismas, varicose veins, etc. More particularly the present invention relates to a box with a regulation mechanism that allows to wind a traction thread into the box, a platform positioned just under the box to stabilize and support the box under stress, and different support systems depending on the organ or anatomical tissue that have to be traction or supported. The system has some accessories to implant the device and the supports in the patient's body.

Many devices have been described to traction organs, like the U.S. Pat. No. 5,474,518 Ner from the same inventors describing a “Corrective device of urinary incontinence in women”, in which an implantable box with a regulation system including a system to be activated from outside the body is disclosed. The system is intended to traction a thread that will reposition the bladder neck.

Another system is the one presented in U.S. Pat. No. 6,117,067 from Gil-Vernet as a “device for the heigh-adjustable fixing and support of internal anatomical organs”. This device comprises an elastic chamber that can move organs by injecting liquid into the chamber through an injection system.

Another system is the one described in U.S. Pat. No, 6,039,686 from Kovac and S. Robert, which describes a “system and method for the long term cure of recurrent urinary female incontinence”. This system includes a urethral traction and anchorage system by a sling and screws fixed to the posterior pubic bone wall.

The exisiting methods that permit the insertion of threads, or slings the intrinsic characteristics of such threads and slings to reposition the bladder or urethra at its original position are nevertheless not quite efficient.

In the above-mentioned U.S. Pat. No. 5,474,518 there are three basic problems:

1—The traction threads have to be knotted one against the other once passed through the regulating box. This drives to potential infections due to bacterial potential inclusion between the knots. Long-term readjustment is difficult due to the fibrosis created in-between the knots that impede the activation of the regulation mechanism.

2—In many patients the traction have to be very strong to reposition determined organs or anatomical tissues. The base of the regulation box have not enough surface area and the traction tension is concentrated in a small surface and the box support tissue gives way loosening the traction support of the hole system.

3—The traction threads can also apply the tension over the tractioned organ or anatomical tissue in a very small surface, damaging and even cutting the organ or anatomical tissue.

The principle disadvantadge of U.S. Pat. No. 6,117,067 is that it do not allow applying traction with enough strength. The strength power of an elastic chamber filled by liquid is limited both in strength and length of traction. The maximum filling of the chamber has a limit and some times is not enough for some patients or to tract some organs that have to be traction during a long way.

The invention of U.S. Pat. No. 6,039,686, even though has a good anchorage to a fix bone support structure, it entails the risk of creating pain and inflammation of the periostium, and also do not allow the readjustment at mid long term of the traction threads.

In all the existing traction systems, the sling that supports the organs or anatomical tissues, have the risk of folding up when being pulled or tensioned at a certain degree. When this happens, the sling is applying tension to an organ or anatomical tissue of a patient, not with all the sling surface, but only by some small parts of it, thus driving to extra tension in some points of the organ or anatomical tissue and creating vessel closure, tissue necrosis and organ or anatomical tissue damage, including orgar or tissue dissection.

For all this reasons, all the existing traction systems have a limited use due to the limited traction strength, and/or the limited length of traction, and/or the limited support base for the tractioning device, and/or limited support systems for the organ or anatomical tissue to be tractioned, and/or the high risk of sling folding when applying tension to it, and/or difficulty in readjusting the tension after the intervention.

The purpose of the present invention is to basically to improve the short and long term functioning of the previous described devices and systems.

This new device comprises:
an implantable box in whose inside housing several mechanical elements to wind in traction threads are housed: a drum with a toothed wheel that gears in a worm;
a fixation screw for the traction threads fixation once passed through the implantable box;
a base support to spread the stress per square inch and stabilization of the traction box against the supportive tissue;
monofilament traction threads that can slide through the anatomical tissue to deliver the traction strength to the sling or the support system used to traction an organ or anatomical tissue;
different systems to subject or support organs or anatomical tissues made of different materials and designs related to the organ or anatomical tissue to be traction;
   this subject or support systems can be:
      a sling, which can incorporate a spread bars to maintain the sling fully open even under traction;
      a traction ball of biocompatible material;
      a silicon coated sling;
      a malleable metallic plate; and
inserting elements to insert the traction system in the patients body.

The implatable regulation box is an improvement of the U.S. Pat. No. 5,474,518, property of the same inventor of the present patent. The proposed improvement is a fixation system for the traction threads to the box, avoiding the external thread knots that remain in the body when using the previous box. Said improvement lies in the incorporation of a fixing screw with a precise design that can be perfectly introduced from the head of the drum in its interior housing, and which function is to immobilize the traction threads tips against the superior part of the head of the drum. So the rotation of said drum shortens the length of the tractioning elements that apply traction to organs or anatomical tissues directly or through other supporting means. To this end, the interior of the drum body has been partially threaded to accept the threads of the fixation screw.

Preferably, the drum will have an interior hollow body with a protruding hollow head provided with two lateral passing ducts to pass the tractioning elements through the head and provided also with a threaded part near the head of the drum to recieve and retain the threads of the fixation screw.

Before, in U.S. Pat. No. 5,474,518, there was any fixation system and the threads were fixed by several knots that remain outside the box, so creating fibrosis, pain and infections.

In the present invention, advantageously, the threads are fixed into the box and there are no thread knots out of the box; therefore improving the readjustment and avoiding patient pain and infections.

Another improvement is the incorporation of a base support or a plataform under the box, which function is to spread the tension in its entire bigger surface, thus delivering the tensions per square inch to the box supportive tissue. This base support has been designed with some holes on it as a pass way for the traction threads to not interfere with the traction threads, and cooperate in the box stabilization or alignment.

The sling subjection mechanism is still traction threads, but in the present invention the connection between the sling and the traction threads includes spreader bars made of biocompatible material to maintain the sling fully extended when under traction. These spreader bars avoid the sling folds, and the support to the organ or anatomical tissue is well distributed by the entire sling surface avoiding tissue damage or section when traction is applied to the sling.

The system of the invention with all this accessories is polyvalent; therefore, it can be used in different parts of the body to tract or support different organs or anatomical tissues. It has different specific accessories for every different organ or anatomical tissue to be supported or repositioned. The organ support accessories can be a sling, a sling with spread bars, a silicone coated sling, a ball, a screw, or a malleable metallic plate.

The system of the invention can be used in the pelvic floor, per incontinence and prolapses, and for this puropose the preferred organ support method is a sling with spread bars to create a fibrosis under the organ or anatomical tissue to be tractioned, maintaining the sling fully extended and giving a wide suport to the tractioned organ. When the system is used to create a “cul the sac” in a flat tissue, like when used to create a neovagina in patients with vaginal agenesia, the preferred support system is a consistent traction ball of a biocompatible material like polyethylene or biocompatible metal. The ball will be tractioned from the regulating box through the traction threads creating a neovagina in a few days, in this case the regulating box will be over the skin and supported in its base by a biocompatible rigid but cushioned plate support, to avoid skin damage and give a strong base to pull the vaginal button.

As previously mentioned, the system can widely be used in different organs or anatomical tissues. It can be used to suport or traction the urethra to solve urinary incontinence in male and female, to support or traction the rectus to solve fecal incontinence, to support or traction the pelvic floor in patients with prolapsus, can be applied to reduce the diameter of the tubes of the digestive tract, to support or reduce the diameter ov the vessels in the vascular system, aneurismas, varicose veins, etc. It can also be used as a traction system for subcutaneous tissue for skin lifting. It can also be used for temporal or definitive bone and or ligament traction, to help in repair of bone fractures or tendon distension or fractures, in tthis application the traction will be made by a biocompatible metal plaque or screws attached to bone. When the traction has to be provisional, a silicon-coated sling can be used to have the traction strength of the sling and the ability of the silicone to slide to be retrieved without surgery.

For its insertion or implantation, some instruments that will be supplied apart to allow the surgeon the decision of using it or not are also object of the present invention. The instrument is basically a needle with a hole in its proximal and distal part to pass the traction thread forward or backwards, and a manipulator to drive the needle.

Additionally, a round shaped biocompatible material to pull an anatomical tissue, creating thus an invagination, a tunnel or a duct can be incorporated.

The activation system for the traction mechanism can be generated by a remote electrical control, a magnetic or a hydraulic system.

The following is descriptive of certain embodiments of the invention. The descriptive cannot be taken in the limited sense, but is made to illustrate the general principles of the invention.

A list of the various references used to describe the embodiments carried out on the apparatus of the present invention follows: traction system (10), supporting means (11), spreader bars (12), spreader bar holes (13), traction threads (14), sling or organ support (15), fixation screw (16), place for the worm (20), place for the drum (21), traction thread passways (22), place for the fixation screw (23), worm (24), thread screw (25), head screw (26), conection system (28), disconnector (29), semi spreader bars (31), semi spreader bar separation (32), drum (33), central hole (34), head of the drum (35), toothed wheel of the drum (36), fixation screw retainer (39), external manipulator (42), manipulator hole (43), external manipulator body (44), inferior duct (47), superior duct (48), reference mark (49), manipulator fixation system (50), first part of manipulator (51), second part of manipulator (52), final part of manipulator (53), manipulator tip (54), lateral ducts (58), needle for traction thread passage (59), manipulator (62), needle tip and proximal holes (64), round box support base (65), holes on the base support (66), rectangular box support base (68), traction button holes (74), holes of the rectangular base support (75), traction box (76), traction buton (77), thread fixation screw (78), central holes (79), central part (80), tractioning elements (81), inserting means (82), and activating means (83).

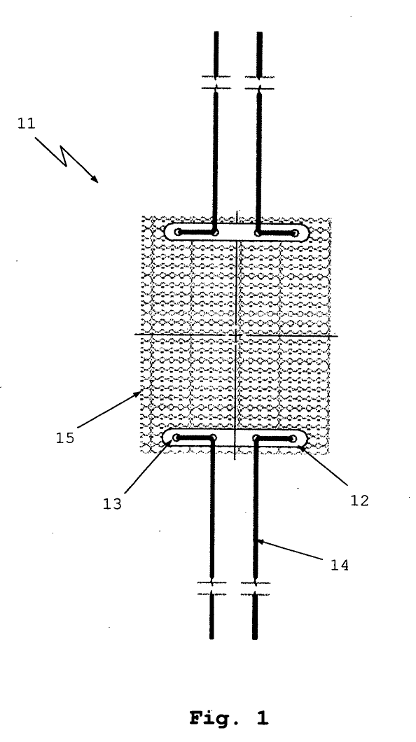

FIG. 1 is an image of the supporting means (11), composed of a polypropylene sling (15) with the spreader bars (12) in each lateral sides, and the traction threads (14) connected to the spreader bars (12) and the sling (15).

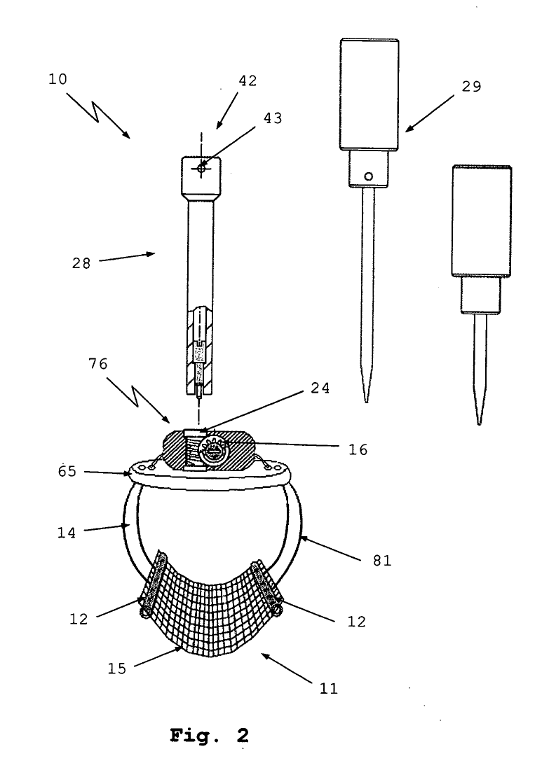

FIG. 2 is a frontal view of the prosthesis (10) with the fixation screw (16) as a regulator system for the traction threads (14), the sling (15) with the spreader bars (12), the external manipulator (42), the disconnector (29) and the screw driver to tight the fixation screw (16).

FIG. 3 is a transversal section of the traction box (76) included in FIG. 2, in which we can appreciate the internal housing design of it.

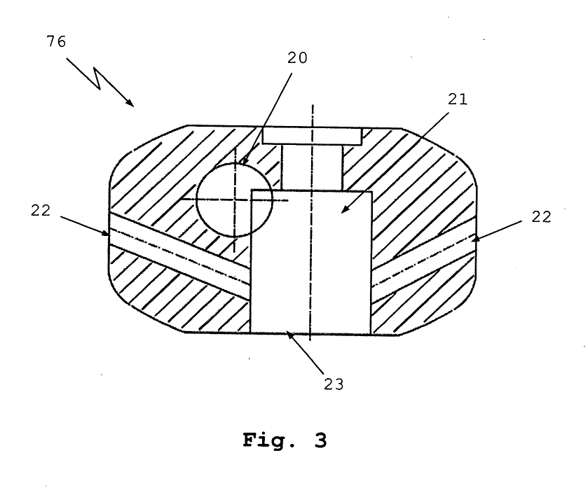

FIG. 4 is a lateral view of the worm (24) that activates or gears the toothed wheel (36) of the drum (33) to wind in the traction threads (14).

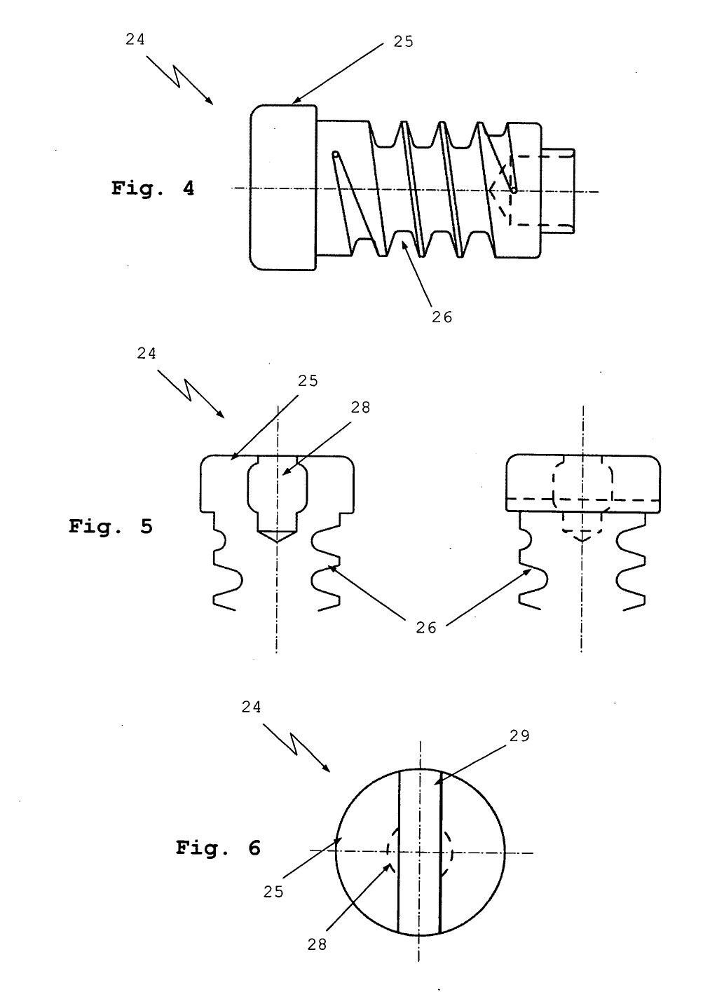

FIG. 5 is a trasversal section of the worm (24).

FIG. 6 is a superior view of the worm (24) with the conection system (28) for the manipulator (42) tip.

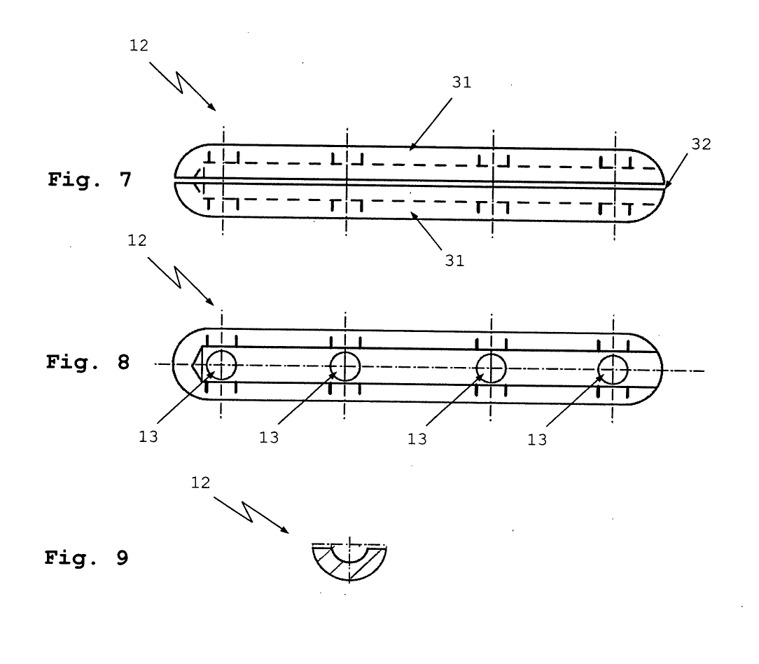

FIG. 7 is a frontal view of the spreader bars (12) placed over and under each lateral side of the sling (15), conforming the spreader support to maintain the sling (15) fully extended under tension.

FIG. 8 is a superior view of a spreader bar (12), including the spreader bars holes (13) to pass the traction threads (14).

FIG. 9 is a transversal section of the spreader bar (12).

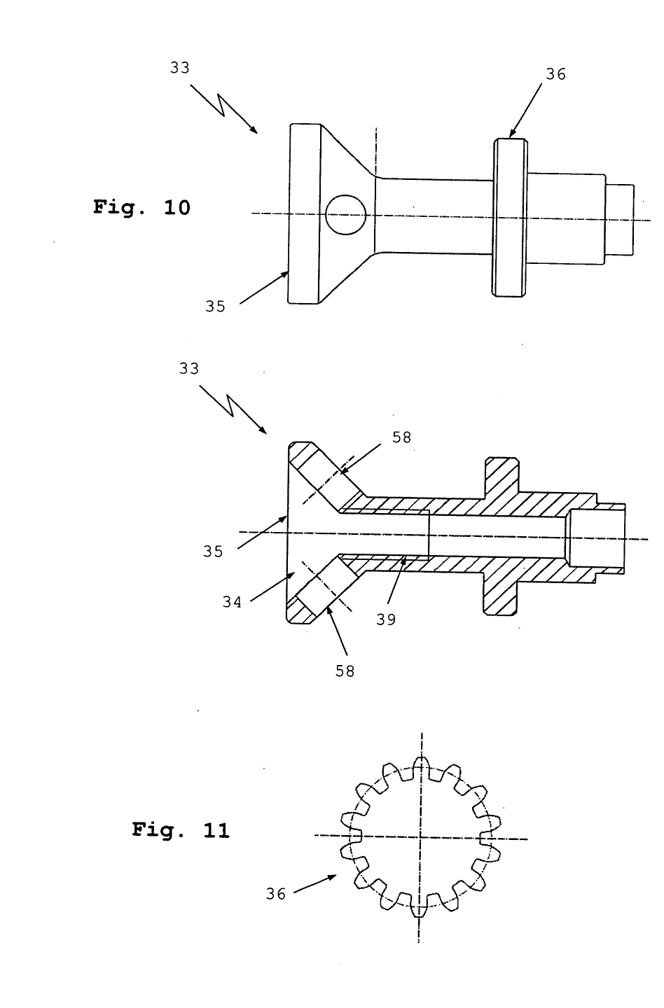

FIG. 10 is a frontal view of the drum (33), with a toothed wheel (36) around the body drum that drives the rotation of the drum (33) to wind the traction thread (14) applying traction to the sling (15).

FIG. 11 is a longitudinal section of the drum (33) including a threaded tunelization or fixation screw retainer (39).

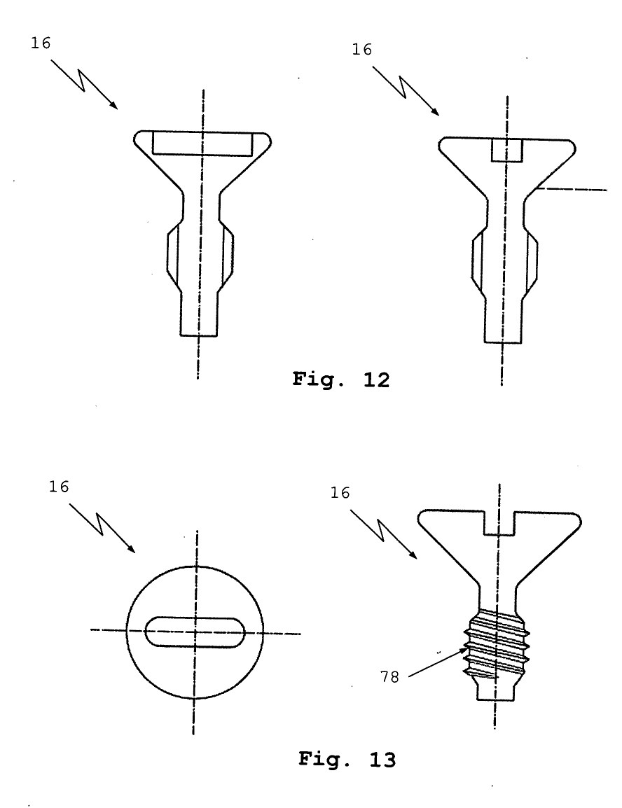

FIG. 12 is a view of the fixation screw (16) in lateral and frontal position.

FIG. 13 is a view of the fixation screw (16) with the threaded body (78).

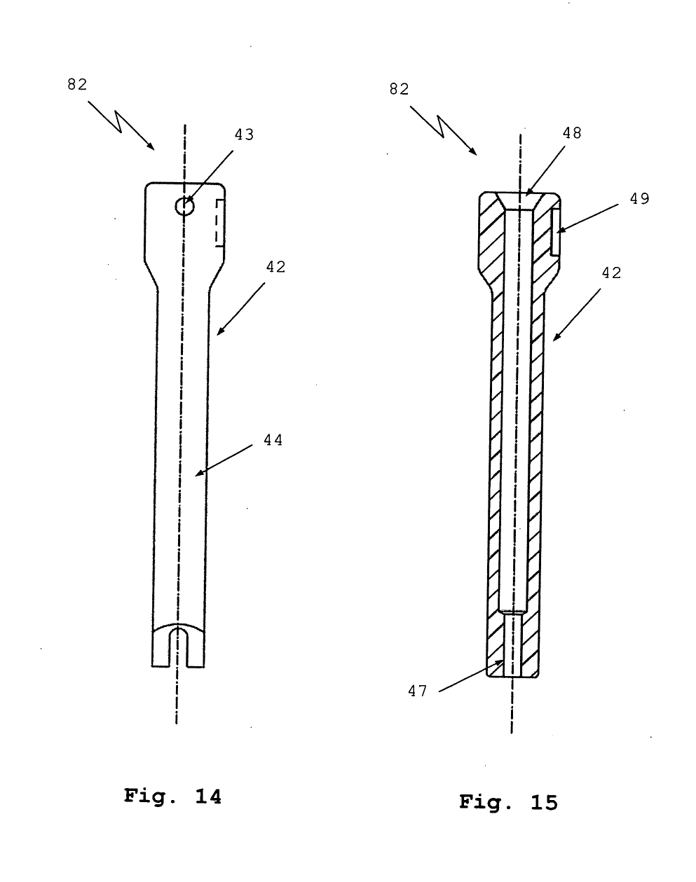

FIG. 14 is a frontal view of the external manipulator (42) with a hole (43) for its fixation to the skin, and a reference mark (49).

FIG. 15 is a longitudinal section of the external manipulator (42) in which we can see that is crossed by a duct (48).

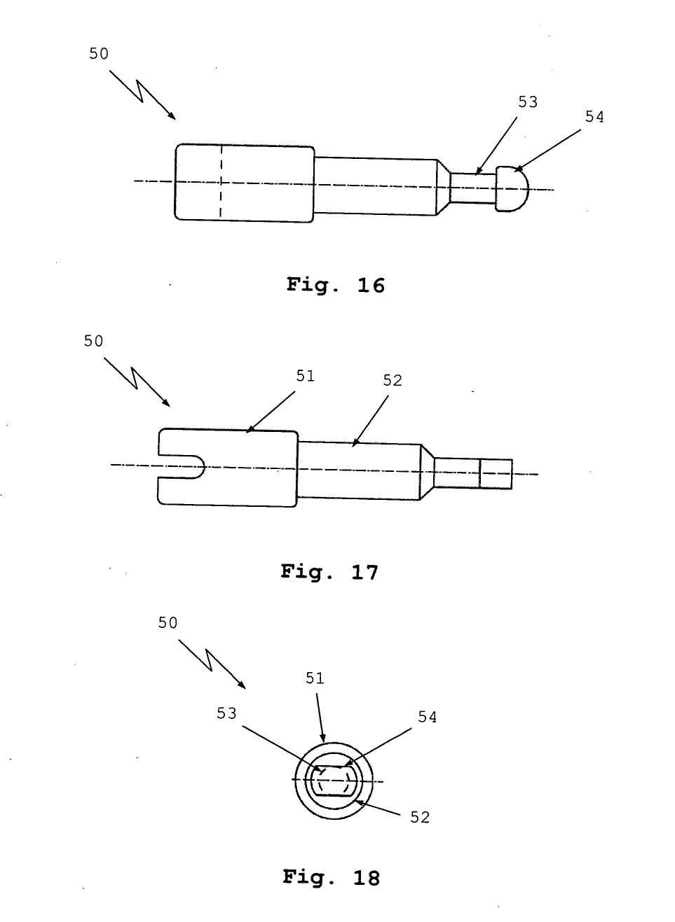

FIG. 16 is a superior view of the manipulator fixation system (50) that is integrated in the internal inferior duct (47) of the manipulator (42).

FIG. 17 is a lateral view of the manipulator fixation system (50).

FIG. 18 is a frontal view of the tip of the external manipulator (42) fixation system (50).

FIG. 19 is a lateral view of the needle (59) used to introduce the traction threads (14).

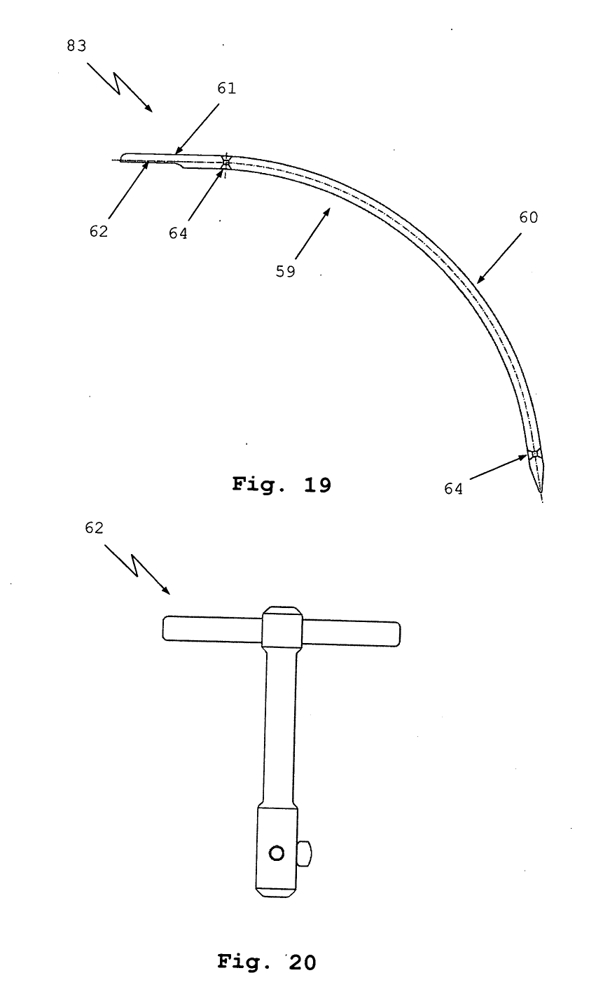

FIG. 20 is a view of the manipulator (62) for the needle (59) used to pass the traction threads (14).

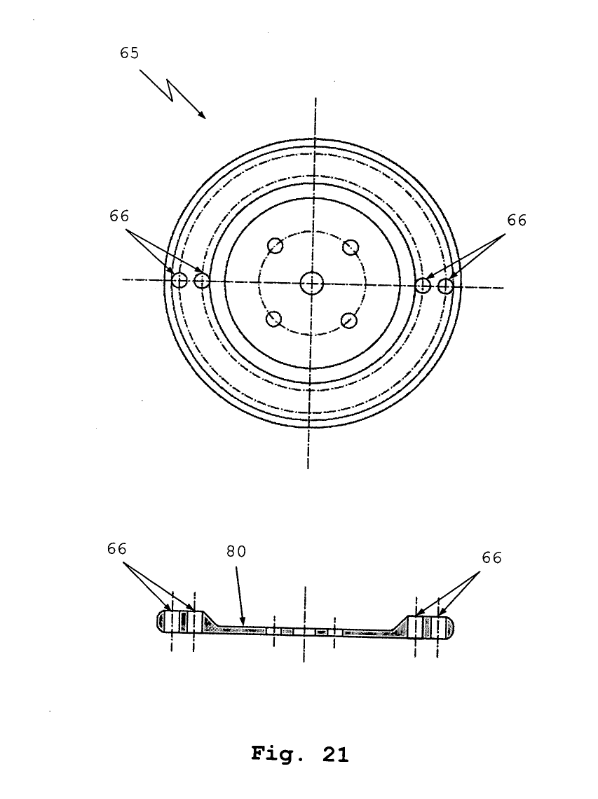

FIG. 21 is a top and lateral view of a first embodiment of the suuport base: a round support base (65) to spread the box (76) stress over tissues under pressure, showing the holes (66) to pass the traction threads (14).

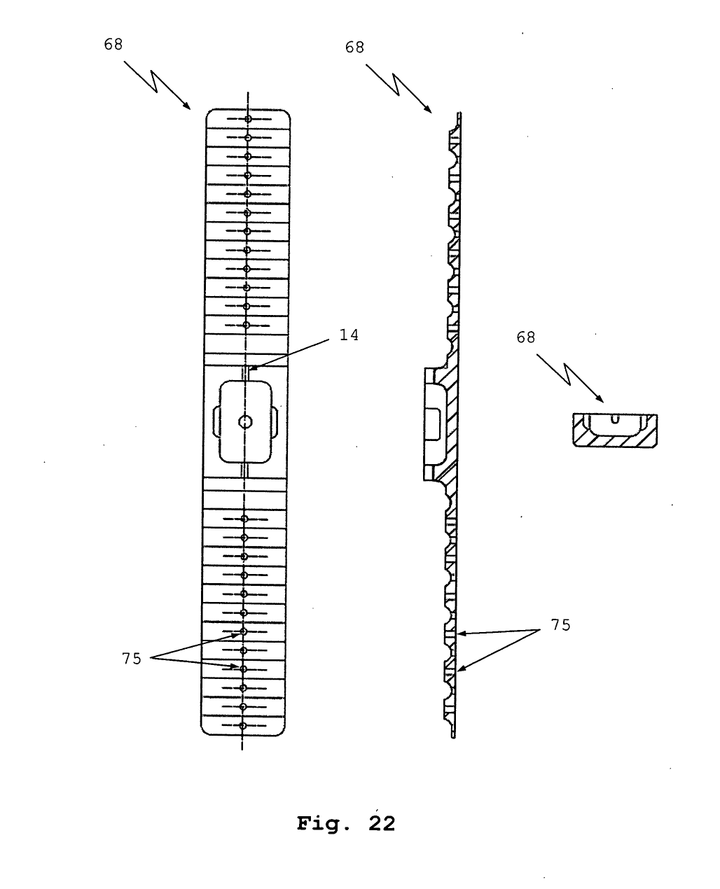

FIG. 22 is a top and lateral view of a second embodiment of the base support: a rectangular base support (68) to spread the box (76) stress over tissues under pressure, showing the holes (75) to pass the traction threads (14).

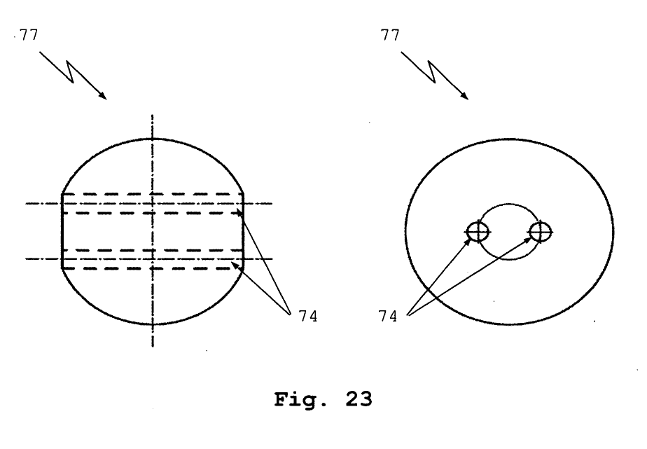

FIG. 23 is a view of a traction button (77) with two passing holes (74) to pass the traction threads (14).

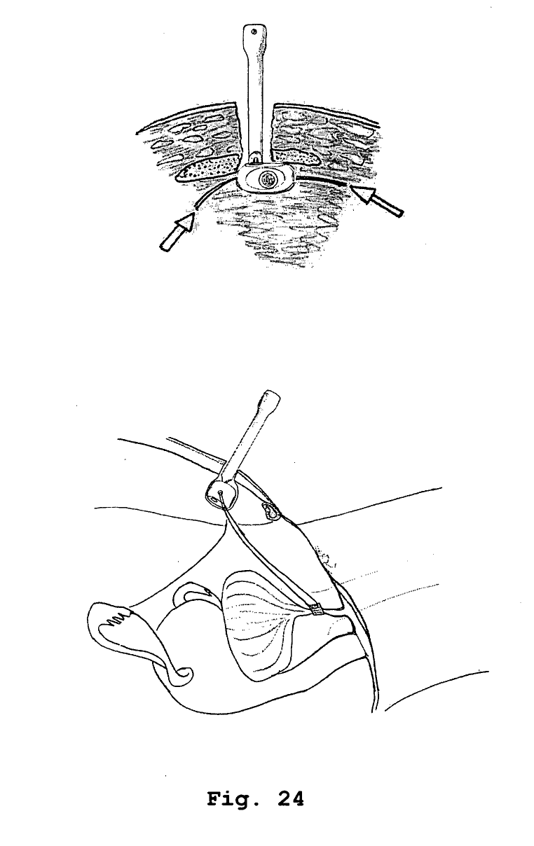

FIG. 24 is a view of the system in its aplication for female incontinence or prolaps.

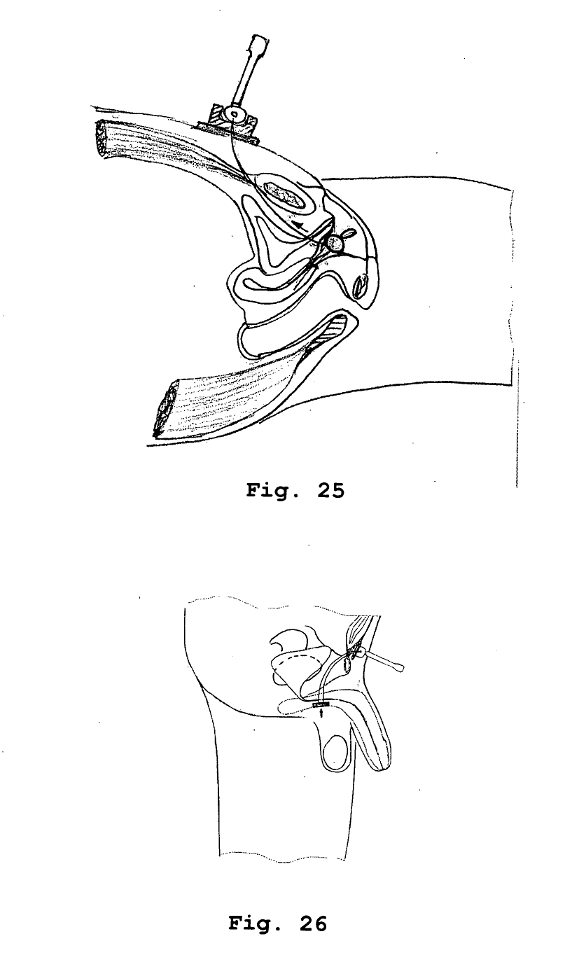

FIG. 25 is a view of the system in its aplication for vaginal agenesis.

FIG. 26 is a view of the system in its apication for male incontinence.

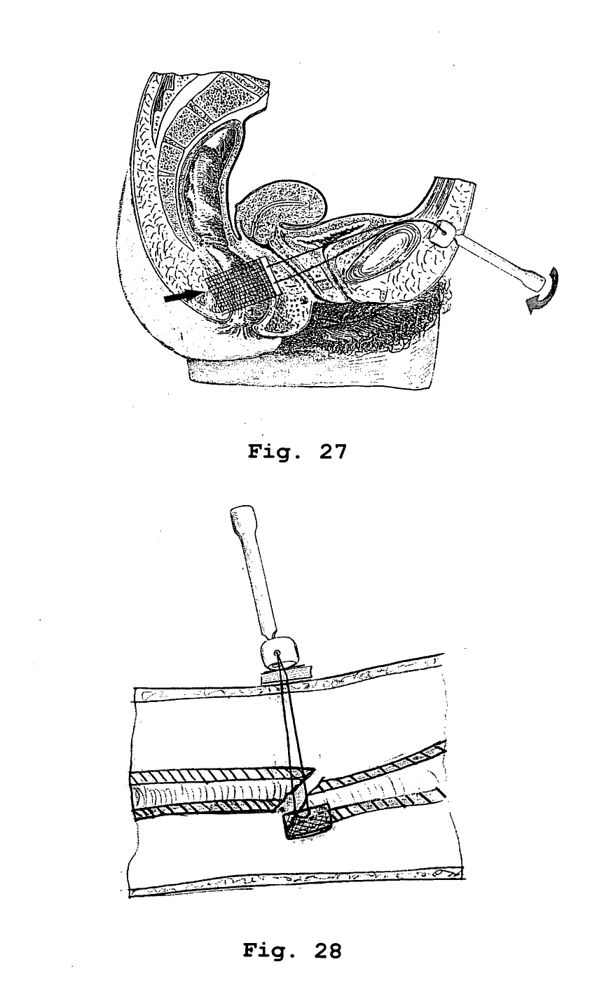

FIG. 27 is a view of the system in its aplication for fecale incontinence.

FIG. 28 is a view of the systyem in its aplication for bone fracture repair.

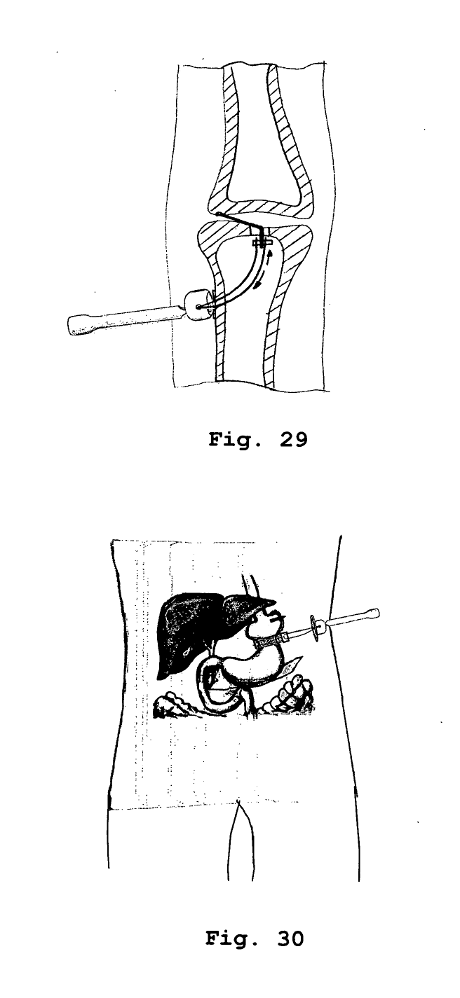

FIG. 29 is a view of the system in its aplication for tendon repair.

FIG. 30 is a view of the system in its aplication for organs or anatomical tubes lumen reduction.

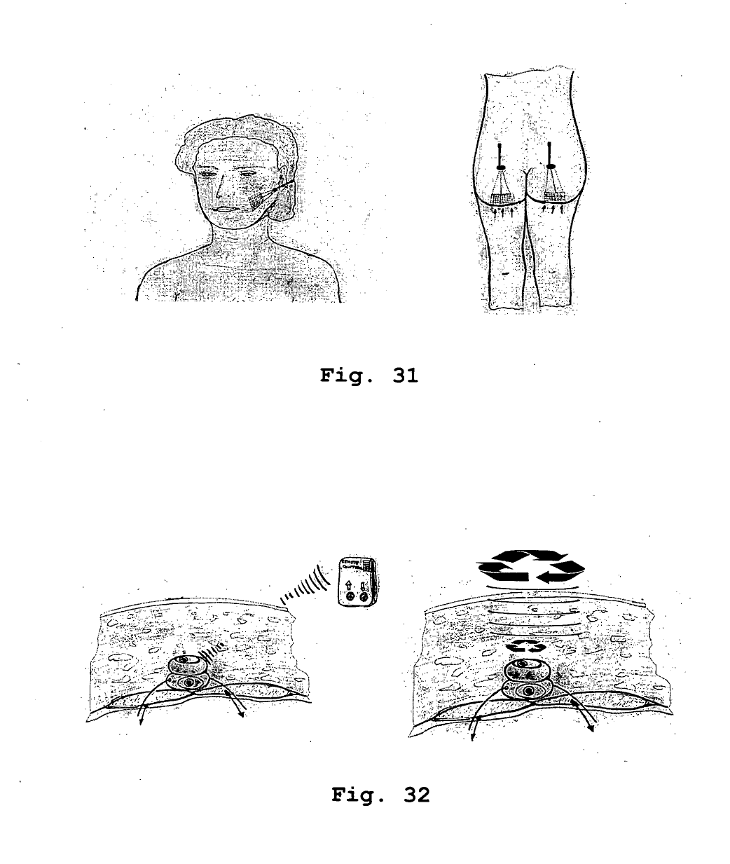

FIG. 31 is a view of the system in its aplication in skin lifting.

FIG. 32 is a view of the system mechanism controled by radiofrecuency or magnetic source.

In one of the preferred embodiments, and as represented in FIG. 2, the system is composed by:
a traction box (76) in which interior there is a traction system to wind into or wind out a tractioning elements (81);
a fixation screw (16) for the tractioning elements (81);
a base support (65) to spread the box (76) stress over the supporting tissue;
tractioning elements (81), such as traction threads (14);
a supporting means (11), such as a sling (15) and sling spreader bars (12); and
an activating means (83), such as an external manipulator (42); and
an inserting means (82), such as a needle (59) and a manipulator (62) to drive said needle (59).

Alternatively, the base support can have different forms and sizes; as a description and not as a limitation can be a rectangular support (68), round support (65) or any other forms and size.

Alternatively, the sling (15) can be changed by different forms and sizes to tract different organs and anatomical tissues, like, as a description and not as a limitation, a vaginal buton (77), see FIG. 23.

The regulation system of the support systems consists of a box (76) with special characteristics shown in FIGS. 2 and 3. In reference to FIG. 3, in the left part of the box (76) the housing (20) to place the worm (24) is represented, and in the right part of the box (76) the place (21) for the drum (33) is represented. In the inferior part of said elongated housing (21), specifically in the housing (23), the fixtion screw (16) is placed by means of introducing it inside the drum (33).

In reference to FIGS. n^{o} 4, 5 and 6, the worm (24) has a thread (26) and a head (25), and in the screw head (25) a connection hole (28) to couple the manipulator (42) tip and the screw (24) is incorporated.

The drum (33), as represented in FIGS. 10 and 11, is made of a single hollow body; and in one side has a conic part (34) in which there are two lateral passing ducts (58) to pass the traction threads (14) through. The traction threads (14) once passed through said ducts (58) are fixed against the conic central hole (34) of the drum (33) by the fixation screw (16), see FIGS. 12 and 13. This fixation screw (16) is introduced into the central hole (34) and it is screwed until its thread (78) is retained by means of the screw retainer (39).

In order to improve the support of the traction box (76), avoiding thus the sinking of the traction box (76) into the supporting tissue as result of the tension generated by the sling tractioning, a box (76) base support (65) has been incorporated to the invention, see FIG. 2. The base support (65) mission is to spread the stress of the inferior part of the traction box (76) and has as a preferred circular configuration with an exterior perimetral part that is higher than the central part (80), as shown in FIG. 21. In this exterior perimetral part four passing holes (66) are arranged in opposite positions, and in the interior part (80) five passing holes (79) are also arranged.

As a preferred design, the inferior part of the traction box (76) is placed against the superior side of the base support (65), and the inferior side of said base support (65) is placed against the supporting tissue that serves as a standpoint for the traction box (76).

The support system for the organ or anatomical tissue to be supported or tractioned can be designed of different compositions and conformations depending on the organ or anatomical tissue to be tractioned. As a preferred design, the support system is composed of a monofilament sling (15) of polypropylene or similar biocompatible material, with a spreader bars (12) at each lateral side of the sling (15) to maintain it fully extended even when is tractioned by the traction threads (14). The spreader bars (12) can be single bars, and as a preferred design the spreader bars (12) are composed by two semi-bars (31) with a separation (32) positioned over and under the lateral sides of the sling (15) and with holes (13) to pass the traction threads (14) that will pull the spreader bars (12) and the sling (15) as a unit when the traction threads (14) are winded into the traction box (76), see FIGS. 1, 7, 8 and 9. The traction box (76) can be supported by a base platform (65) to spread the tension of the traction box (76) over the supporting tissue.

In case the system (10) is used to create a neovagina, by tractioning the vaginal flat membrain, the support system to pull the flat vaginal membrane is a round ball, not represented in the drawings, with holes to pass the traction threads (14) through it and connect the traction threads (14) to the regulation box (76). As shown in FIG. 22, the regulation box (76) will be placed over a rectangular base support (68) that in this case will be over the skin of the patient, and will have an elongated rectangular shape including a plurality of holes (75) along its body to pass the traction threads (14) and a cushion of a soft material, not shown in the figures, positioned between the rectangular base support (68) and the patient skin. The mission of the cushion is to avoid lesions in the skin of the patient due to the continuous pressure of the regulation box (76) over the base support (68) and said base support (68) over the skin.

In case the system (10) is used to pull a bone or ligament to repair a traumatology problem, the base support (68) can be one of the previous described: 65 or 68, implanted or over the skin, and the bone or ligament support will be a silicone coated sling (15), to maintain the mechanical strength of the sling (15), and to be able to slide the sling out of the body once the bone or the ligament is repaired. To slide the sling (15) out of the body, the traction thread (14) will be cutted and by pulling thje traction thread (14) out, the silicone-coated sling (15) will slide out of the body.

In case the system is used to pull subcutaneous tissue in esthetic repairs, the sling (15) will be attached only by one side to the traction thread (14). The sling (15) ill integrate in the subcutaneous tissue and once integrated, will be pulled accordingly by the traction threads (14) to lift the skin.

In case the system (10) is used to maintain or decrease the lumen of a tube of the digestive or the vascular system, the tissue support sling (11) will be a sling that can be coated with silicone, and the sling can be pulled by the traction threads (14) directly to the regulation box (76) or indirectly crossing the traction threads to a rectangular base support (68) system of the traction box (76) with holes to close the lumen or the tube like with a sphyncter.

In order to pass the traction threads (14) a needle (59) with a hole in the proximal and a hole in the distal part can be used by placing said traction threads (14) through the holes (64) and by pulling the needle (59), the traction threads (14) will follow the needle way into the body. The needle is provided with a manipulator (62) located in the proximal part to help the surgeon drive the needle tip direction and push it accordingly into the body, see FIGS. 19 and 20.

In order to activate the regulation mechanism insided the box (76), a manipulator (42) can be connected to the box (76). Said manipulator (42) will have a hole (43) in the proximal part to place a suture thread to be able to localice the manipulator (42) if it sinks into the abdomen or other part of the body, see FIG. 2.

In reference to FIGS. n^{o} 14 and 15, the external manipulator (42) has an elongated body (44) and it incorporates in its proximal end a hole (43) for its fixation to the skeen and also a reference mark (49). The manipulator (42) along all its length is crossed internally by a duct (48), which ends in an inferior duct (47).

The manipulator (42) can be replaced by an electronic or magnetic or hydraulic mechanism that will also activate the regulating box (76) internal mechanism from outside the body.

Referring now to FIGS. n^{o} 16, 17 and 18, the manipulator fixation system (50) that is integrated in the internal duct (47) of the manipulator (42) has a first cylindrical body (51), a second cylindrical body (52) with a smaller diameter, a final cylindrical body (53) and a tip (54), which has a convex configuration.

Accordingly, the scope of the present invention is not limited to the specific embodiment as illustrated herein, but is limited only by the following claims and equivalents thereof.

1. “Implantable mechanical device to apply traction to organs or anatomical tissues for its repositioning during and after a surgical intervention”, comprise an implantable box in which interior there are a housing to place a drum with a toothed wheel connected to said drum and a worm gear, being said worm gear in meshing with said toothed wheel, and said box having two lateral ducts to pass the the tractioning elements through and being connected to said drum; and an external manipulator for coupling it to the head of the worm to transmit the actuating force, wherein it furthermore comprises:
a fixation screw (16) within the box (76) in order to immobilize the tractioning elements (81) tips against the superior part of the head (35) of the drum (33), having said drum (33) an interior hollow body with a protruding hollow head (35) provided with two lateral passing ducts (58) to pass the tractioning elements (81) through said head (35) and a threaded part (39) near the head of the drum to recieve and retain the threads (78) of the fixation screw (16); having said fixation screw (16) the precise dimensions to fit perfectly in the interior hollow of the drum (33).
a base support (65) located under the box (76) to spread the box (76) stress over the supporting tissue;
inserting means (82) to insert the traction device (10) in the patients body;
activating means (83) to allow the activation of the device (10) from outside the body; and
supporting means (11) to suport the target organ.

2. “Implantable Implantable mechanical device to apply traction to organs or anatomical tissues for its repositioning during and after a surgical intervention”, according to claim 1, the activating means (83) comprise a remote electrical control, a magnetic or a hydraulic system.

3. “Implantable mechanical device to apply traction to organs or anatomical tissues for its repositioning during and after a surgical intervention”, according to claim 1, wherein the supporting means (11) comprise a biocompatible sling (15) that can incorporate at least two spread bars (12), on in each lateral side of the sling (15), with a plurality of holes (13) to pass the tractioning elements (81) and maintain the sling (15) fully open even under traction.

4. “Implantable mechanical device to apply traction to organs or anatomical tissues for its repositioning during and after a surgical intervention”, according to claim 1, wherein the supporting means (11) comprise a traction ball of biocompatible material.

5. “Implantable mechanical device to apply traction to organs or anatomical tissues for its repositioning during and after a surgical intervention”, according to claim 1, wherein the supporting means (11) comprise a malleable metallic plate.

6. “Implantable mechanical device to apply traction to organs or anatomical tissues for its repositioning during and after a surgical intervention”, according to claim 3, wherein the spreader bars (12) of the the supporting means (11) are made of biocompatible material.

7. “Implantable mechanical device to apply traction to organs or anatomical tissues for its repositioning during and after a surgical intervention”, according to claim 3, wherein the sling (15) is silicone-coated.

8. “Implantable mechanical device to apply traction to organs or anatomical tissues for its repositioning during and after a surgical intervention”, according to claim 3, wherein the sling (15) is connected to a traction thread (14) only by one side to pull subcutaneous integrated tissue as a skin lifting system.

9. “Implantable mechanical device to apply traction to organs or anatomical tissues for its repositioning during and after a surgical intervention”, according to claim 8, wherein the base support (65) incorporates several holes (66) on it as a pass way for the traction threads (14) to not interfere with said traction threads (14).

10. “Implantable mechanical device to apply traction to organs or anatomical tissues for its repositioning during and after a surgical intervention”, according to claim 1, wherein the base support (65) has a circular configuration with an exterior perimetral part higher than the central part (80), being arranged in said exterior perimetral part at least two passing holes (66) in opposite positions, and being arranged in the interior part (80) at least one passing hole (79).

11. “Implantable mechanical device to apply traction to organs or anatomical tissues for its repositioning during and after a surgical intervention”, according to claim 9, wherein the base support (65) has a rectangular elongateed configuration including a plurality of holes (75) along its body to pass the traction threads (14) and a cushion of a soft material positioned between the base support (68) and the patient skin.

12. “Implantable mechanical device to apply traction to organs or anatomical tissues for its repositioning during and after a surgical intervention”, according to claim 1, wherein the inserting means (82) comprise a needle (59) with a hole (64) in its proximal part and another hole (64) in its distal part to pass the traction thread (14) forward or backwards, and said needle (59) provided with a manipulator (62) in its proximal end to drive said needle (59) tip direction by the surgeon.

13. “Implantable mechanical device to apply traction to organs or anatomical tissues for its repositioning during and after a surgical intervention”, according to claim 1, wherein additionally a round shaped biocompatible material to pull an anatomical tissue, creating thus an invagination, a tunnel or a duct can be incorporated.

14. “Implantable mechanical device to apply traction to organs or anatomical tissues for its repositioning during and after a surgical intervention”, according to claim 1, wherein the organs or anatomical tissues target are pelvic floor organs or anatomical tissues support or traction.

15. “Implantable mechanical device to apply traction to organs or anatomical tissues for its repositioning during and after a surgical intervention”, according to claim 1, wherein the organs or anatomical tissues target are bone or tendons support or traction.

16. “Implantable mechanical device to apply traction to organs or anatomical tissues for its repositioning during and after a surgical intervention”, according TO claim 1, wherein the organs or anatomical tissues target are digestive or vascular or urinary or reproductive organs or ducts support or traction.

17. “Implantable mechanical device to apply traction to organs or anatomical tissues for its repositioning during and after a surgical intervention”, according to claim 1, wherein the organs or anatomical tissues target are animals organs or anatomical tissues.
